(19) United States
(12) Patent Application Publication
(10) Pub. No.: US 2017/0368317 A1
(43) Pub. Date: Dec. 28, 2017

(54) TORQUE DEVICES

(71) Applicant: The Board of Trustees of the Leland Stanford Junior University, Stanford, CA (US)

(72) Inventors: Torbjörn Lundh, Billdal (SE); Oliver Oppers Aalami, Palo Alto, CA (US); Nathan Koji Itoga, Menlo Park, CA (US)

(21) Appl. No.: 15/636,246

(22) Filed: Jun. 28, 2017

Related U.S. Application Data
(60) Provisional application No. 62/355,770, filed on Jun. 28, 2016.

(51) Int. Cl. A61M 25/09

(52) U.S. Cl. CPC A61M 25/09041; A61M 2025/09116; A61M 2025/09125

(57) ABSTRACT

Torque devices for navigating a guidewire through a body lumen are disclosed. The devices have a variable speed transmission design, including at least a first transmission region along a first position of the device, and a second transmission region along a second position of the device. The first and second transmission regions have different diameters, thereby allowing rotational control of the guidewire between at least two different stroke angles.

### PRIORITY AND CROSS REFERENCE TO RELATED APPLICATIONS

This application claims the benefit of U.S. Provisional Application No. 62/355,770, filed on Jun. 28, 2016, which is hereby incorporated by reference in its entirety.

### BACKGROUND

### Field

The present disclosure relates generally to devices, methods, and/or kits for guidewire- and/or catheter-based interventions.

### Description of the Related Art

Endovascular procedures include diagnostic and/or therapeutic interventions that span over a variety of subspecialties of the broad field of vascular surgery. During an endovascular procedure specific vessels are targeted by using guidewires and catheters. As targeted vessels differ by caliber, flexibility and tortuosity, a variety of guidewires and variously shaped catheters are used to maneuver a guidewire from the initial access point to the vessel of interest (e.g., placing a guidewire in the femoral artery and guiding it into the coronary artery in the case of a coronary intervention).

### SUMMARY

In some embodiments, a torque device for better gripping and navigating a guidewire through a body lumen, the torque device comprising an elongate body with a through channel and a locking mechanism, which together are configured to reversibly engage the guidewire within the torque device, wherein the elongate body comprises a variable rotation speed transmission, comprising at least first and second transmission regions, the first transmission region having a first diameter and a first longitudinal position along the elongate body, and the second transmission region having a second diameter and a second longitudinal position along the elongate body, wherein rotation of the first transmission region by manual operation is configured to induce rotation of an engaged guidewire at a first stroke angle, and rotation of the second transmission region by manual operation is configured to induce rotation of an engaged guidewire at a second stroke angle, which is different from the first stroke angle. In some embodiments of the device, the at least first and second transmission regions are configured to be manually operated independently of each other.

In some embodiments of the device, the stroke angle of the engaged guidewire can be varied between the first and second stoke angles by shifting between manual rotation of the first and second transmission regions, respectively.

In some embodiments of the device, at least two stoke angles allow for at least two levels of rotational control of the engaged guidewire.

In some embodiments of the device, a first level of rotational control is configured to rotate the engaged guidewire at a first rotation radius, and a second level of rotational control is configured to rotate the guidewire at a second rotation radius.

In some embodiments of the device, the first rotation radius is configured for a lower degree of rotation of the guidewire and the second level of rotation is configured for a higher degree of rotation of the guidewire. In some embodiments, the device is configured to provide a stroke angle at least three times the stroke angle of traditional torque devices.

In some embodiments, a method of navigating a guidewire through a body lumen, the method comprising providing the torque device, engaging the guidewire within the through channel by actuating the locking mechanism, manually rotating at least one of the transmission regions thereby inducing rotation of the engaged guidewire at a stroke angle, and navigating the guidewire through the body lumen.

In some embodiments of the method, switching from the manual rotation of the first transmission region to manual rotation of the second transmission region, causes the stroke angle to shift from the first stoke angle to the second stroke angle.

In some embodiments of the method, the device provides the option for a stroke angle at least three times the stroke angle of traditional torque devices.

In some embodiments, a kit for navigating a guidewire through a body lumen, the kit comprising the torque device and a guidewire is provided.

### BRIEF DESCRIPTION OF THE DRAWINGS

FIG. 1 shows a stroke length (SL) indicated by the double-headed arrow.

FIG. 2 shows several views of an embodiment of the torque device of the present disclosure.

FIG. 3 shows an embodiment of a guidewire with a curved distal tip.

FIG. 4 shows the state-of-the-art conventional torque device Terumo.

FIG. 5 shows alternative embodiments of the torque device with a “Screw Cap” of the present disclosure.

FIG. 6 shows how an embodiment of the device according to the present disclosure can be locked and unlocked to the wire using only one hand.

FIG. 7 shows an alternative embodiment with a “Screw Cap” design.

FIG. 8 shows an embodiment of the Screw Cap design without the Screw Cap locking mechanism.

FIG. 9 shows an alternative embodiment with a “Slider Cap” design.

FIG. 10 shows some contemplated alternative embodiments of the Slider Cap (FIG. 9; left) that can slide back-and-forth to allow the guidewire to be released or fixed with a one hand grip.

FIG. 11 shows the experimental setup of benchtop performance testing of Example 1.

FIG. 12 shows two snapshots of the benchtop performance testing of Example 1.

FIG. 13 shows an embodiment of the torque device of the present disclosure with a “flared” design of flexible prongs.

FIG. 14 shows another embodiment of the torque device of the present disclosure with a “flared” design of flexible prongs.

### DETAILED DESCRIPTION

Utilizing catheters and guidewires during endovascular procedures allow minimally invasive diagnostic and therapeutic interventions across a diversity of specialties including, but not limited to, cardiology, radiology, neurosurgery and vascular surgery. For example, vascular surgery is a surgical subspecialty in which diseases of the vascular system are managed by, without limitations, minimally-invasive catheter-based procedures.

As used herein, “vascular system,” “vasculature,” “vascular,” “endovascular” refers to the components of the circulatory and lymphatic system comprising arteries, veins, and vessels of the lymphatic circulation, bone vasculature, etc.

One of the initial steps of catheter based procedures is putting in place a “highway” for all planned interventions which involves navigating and placing a guidewire to a distal target across tortuous and narrow lesions and selecting target vessels past multiple branch points. Guidewires facilitate the delivery of a wide variety of catheters, stents, balloons, other interventional devices, therapeutic compositions, pharmaceutical formulations, drugs, etc. to a procedure site within a subject.

Worldwide millions of endovascular procedures are performed. Torque devices are used in up to an estimated three quarters of endovascular procedures. The torque device, like guidewires and catheters are disposable to ensure sterility between operations as they are in direct contact with a subject's bodily fluids.

Endovascular specialists are challenged on a daily basis to navigate the vasculature in a subject's body, for example, blood vessels, lymphatic vessels, etc. The goal is to navigate one or more guidewires through one or more vessels in the body and reach a location of a pertinent disease that needs treatment. The navigation path through the one or more vessels is not always straightforward. For example, a surgeon may have to navigate a guidewire through several bifurcations in a blood vessel before reaching a desired location or an entrance point of the desired bifurcation.

To help guide a guidewire to the right place, endovascular specialists use special guidewires and catheters with different curves (curvatures) and varying stiffnesses. A drawback of traditionally used guidewires is the lack of a “response” at the distal tip (end) of the guidewire. This can be a major limitation as guidewires are used in practically every endovascular case.

Traditionally, directional navigation is achieved by pointing a curved guidewire using a “torque device” which is frequently attached to the distal end of the guidewire by a screwing mechanism, whereby the torque device needs to be removed and reattached at the end of the guidewire after each guidewire and catheter exchange.

Currently used torque devices aim to provide the operator a good grip of the guidewire in order to navigate and steer the guidewire through the vasculature. However, surgeons are often faced with tortuous anatomy and challenging vascular branches which are difficult to navigate. In addition, when the walls of the vessels are diseased (for e.g., with atherosclerotic plaques), continuous movement of the guidewire tip is required to remain within the lumen of the vessel.

Torque devices are used to manipulate guidewires. However, a way to improve maneuverability of the guidewire was sought as current torque devices used to manipulate guidewires allow for only limited angular rotation of the guidewires.

Thus, some embodiments of the present disclosure relate to addressing the need to improve maneuverability and provide a mechanism to help with guidewire tip rotation to pass complex lesions.

In some embodiments, the present disclosure provides torque devices, methods, and/or kits for guidewire handling. In some embodiments, the present disclosure provides torque devices, methods, and/or kits for endovascular guidewire handling. Therefore, in some embodiments, the present disclosure provides torque devices, methods, and/or kits for non-endovascular guidewire handling.

Non-limiting examples of guidewires include glide guidewire, k-wire (used in spinal surgery to penetrate a bone at a precise spot), steel wires, nitinol wires, braided wires, silicone-coated wires, polytetrafluoroethylene-coated wires, etc.

In some embodiments, advantage is taken of humans' lifelong training in rolling thin cylindrical like objects between our thumbs and fingertips like crayons, pencils, pens, branches, strings, etc. Thus, humans have an almost intuitive perception of the rolling and the resulting turning. In some embodiments, the torque device is designed to keep the one-to-one connection between the finger and thumb tips and the tip of the catheter.

Using SolidWorks and 3D printing, an array of embodiments were tested to develop a design of a torque device. Thus, a novel torque device for guidewires was developed. In some embodiments, a novel torque device is provided. In some embodiments, a novel torque device for guidewires is provided. In some embodiments, a torque device with at least one radius is provided. In some embodiments, a torque device with more than one radius is provided. In some embodiments, a torque device with multiple radii is provided.

In order to make a torque device efficient, a user of the torque device would like to be able to make at least one complete 360 degree (radial angle) turn for every fingertip “stroke.” The greater the radial angle that can be covered by a single fingertip stroke, the faster a user can search for and/or reach a desired location, for example, the entrance point to a desired bifurcation in a vessel.

The present disclosure provides embodiments of a simple multi radius torque device to be used in conjunction with a guidewire to improve the speed and efficiency when navigating vessels and selecting branch vessels during any endovascular procedure. Such a torque device can be used in almost all endovascular procedures. It will be appreciated by one of ordinary skill in the art that the multi radius torque device according to the present disclosure can be adapted for use in conjunction with any type of guidewire.

In some embodiments, the torque device allows for multiple levels of rotational control. In some embodiments, the torque device has a rotational control with a larger radius to provide a more controlled but lower degree of rotation of a guidewire. In some embodiments, the torque device has a rotational control with a smaller radius to provide a higher degree of rotation of a guidewire. In some embodiments, the torque device especially provides a small enough radius to substantially increase the level of rotation of a guidewire.

In some embodiments, the torque device has a variable transmission torque design, i.e., a variable transmission torque design allows for rotational control over a large radius and/or over a small radius. In some embodiments, a variable transmission torque design of the torque device design allows a guidewire to be controlled at different rotational levels. In some embodiments, different diameters on the torque device provide different “gears” to vary the rotational amplitude.

In some embodiments, the larger radius range is referred to as a first rotation radius. In some embodiments, the larger radius range is about 1 mm to about 10 mm. In some embodiments, the larger radius range is about 2 mm to about 6 mm. In some embodiments, the larger radius range is about 2.5 mm to about 5 mm. In some embodiments, the larger radius range is about 3 mm to about 4 mm. In some embodiments, the larger radius is about 0.5, 1, 2, 3, 4, 5, 6, 7, 8, 9, 10, 11, 12, 13, 14, 15, 16, 17, 18, 19, or 20 mm, or a value within a range defined by any two of the aforementioned values

In some embodiments, the smaller radius range is referred to as a second rotation radius. In some embodiments, the smaller radius range is about 0.4 mm to about 5 mm. In some embodiments, the smaller radius range is about 0.5 mm to about 2 mm. In some embodiments, the smaller radius range is about 0.75 mm to about 1.85 mm. In some embodiments, the smaller radius range is about 1 mm to about 1.75 mm. In some embodiments, the smaller radius is about 0.2, 0.4, 0.6, 0.8, 1, 2, 3, 4, 5, 6, 7, 8, 9, or 10 mm, or a value within a range defined by any two of the aforementioned values.

In some embodiments, the novel torque device provides control over a large radius. In some embodiments, the novel torque device provides control over a small radius. In some embodiments, the novel torque device provides control over a large radius or a small radius. In some embodiments, the novel torque device provides control over a large radius and a small radius. In some embodiments, the novel torque device provides control over a large plurality of radii.

A more concrete comparison between the simple design of the torque device of the present disclosure and the current state-of-the-art may be appreciated by a review of U.S. Pat. No. 5,392,778, which attempts to address the problem of increasing the torque amplitude. However, U.S. Pat. No. 5,392,778 attempts to address the problem by suggesting a complicated system of real gears that could optionally be motorized. U.S. Pat. No. 5,392,778 is hereby incorporated by reference in its entirety.

As torque devices should be cheap to manufacture, an advantage of the design of the device of the present disclosure is that there is minimal to no added cost compared to current torque devices being used. Furthermore, given that torque devices are preferably one-time use only (disposable) consumables, it is imperative to provide a cheap yet sophisticated torque device.

Non-limiting advantages of the torque device of the present disclosure include improved ability to more efficiently navigate and select for branch-off entrance points in vessels, safer, quicker and hence also more cost effective catheter procedures, decreased operating time leading to savings in medical expenses, etc.

Even though motorized and mechanically geared torque devices exist, humans are accustomed to rolling cylindrical shaped object between their fingers and thumbs providing direct tactile perception of the cylindrical object in hand.

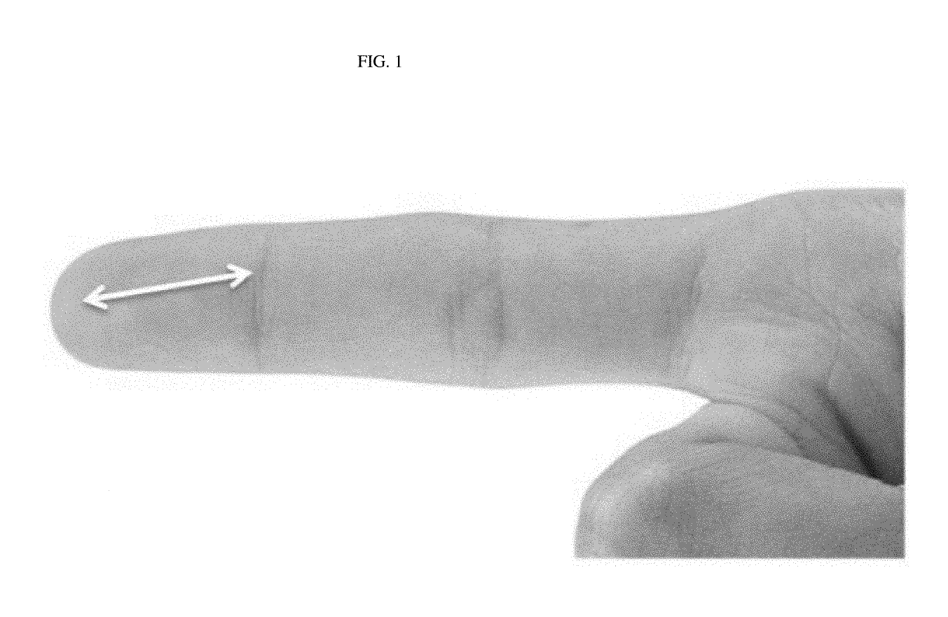

The embodiments of the torque device according to the present disclosure take advantage of this tactile perception. The rolling action can be quantified in terms of stroke length (SL), indicated in FIG. 1 by the double-headed arrow, which is defined as the maximal length a torque device can be rolled at the most distal phalange of a pointer finger. To get a general estimate, it can be assumed that a general maximal SL is about 25 mm (based on the experiment results shown in Table 1 for a sample of different stroke lengths).

The SL is transformed into a stroke angle (SA). SA=SL/(2πr), where r is the radius of the torque device. SA is the maximal angle (measured in whole turns) a torque device will turn in a single stroke.

To make the radius small enough such that the curved tip of the guidewire can reach around in every direction without changing the finger grip, an r<SL/(2π) is required.

In order to perform a vessel bifurcation search, one needs to determine how quickly one can advance a guidewire along a blood vessel to find a bifurcation in the vessel by probing the curved guidewire tip advancing along the inside wall of the vessel. Thus, in some embodiments, in order to perform a vessel bifurcation search, one needs to determine the maximum velocity at which one can advance a guidewire, for example, along a blood vessel to find a bifurcation in the vessel by probing the curved guidewire tip advancing along the inside wall of the vessel.

A mathematical formula can be used to compute how fast or the velocity at which one can advance through a mother vessel while searching for a daughter branch. If a daughter vessel has an opening diameter of d mm, assuming that the stroke length is SL mm, the torque device has a radius of r mm, and the stroke frequency is f Hz, one can advance with a maximum velocity of SL×d×f/(2πr) mm/s if the strokes are made in the same direction. It is noted that if the strokes are made in a “back-and-forth” manner and SL<2πr−d, then one may completely miss the branch no matter how slow one advances. The advancement velocity for a vessel bifurcation search can be calculated as, for example, in Example 2. Therefore, if the torque device radius is reduced by 50%, the search time will be reduced by the same factor.

Thus, a small radius of the torque device gives a high stroke angle. But a too small radius will imply: (1) less leverage for torque strength; (2) less easy to handle in a “tweezers grip” (that's why torques devices are used in the first place); (3) less precision for small angle movements; (4) less contact area with the finger tips and hence lesser friction. Therefore, it is desirable to have both a small and a large radius available in the same torque device at the same time. This way the operator has a choice to pick the optimal radius for the situation at hand.

In some embodiments, the novel torque device features two or more levels of radii. In some embodiments, at least one larger radius is for strength and fine-tuning the guidewire. In some embodiments, at least one smaller radius is for maximal angular stroke.

In some embodiments, a torque device is provided that has an elongate body with a through channel. In some embodiments, a torque device comprising a variable speed transmission design is provided. In some embodiments, the variable speed transmission comprises at least two transmission regions. In some embodiments, the variable speed transmission comprises a region of first transmission and a region of second transmission. In some embodiments, the first transmission region has a first diameter and a first longitudinal position along the elongate body, and the second transmission region has a second diameter and a second longitudinal position along the elongate body.

The length of the elongate body can range from about 1 inch to about 3 inches. In some embodiments, the length of the elongate body is about 1, 1.25, 1.5, 1.75, 2, 2.25, 2.5, 2.75, 3, 3.25, 3.5, 3.75, or 4 inches, or a value within a range defined by any two of the aforementioned values. In some embodiments, the diameter of the through channel ranges from about 0.01 inch to about 0.075 inch. In some embodiments, the diameter of the through channel is about 0.0025, 0.005, 0.01, 0.014, 0.018, 0.025, 0.03, 0.035, 0.04, 0.045, 0.05, 0.055, 0.06, 0.065, 0.07, 0.075, 0.08, 0.085, 0.09, 0.095, or 0.1 inch, or a value within a range defined by any two of the aforementioned values.

In some embodiments, the torque device has a locking/grasping mechanism, which allows the reversible engagement of a guidewire within the through channel of the elongate body of the torque device.

In some embodiments, the torque device allows a user to change the stroke angle by simply changing the position of their fingers on the torque device. Thus, in some embodiments, the torque device allows the user to attain at least two stroke angles by simply changing the position of their fingers on the torque device. In some embodiments, the torque device allows the user to attain more than two stroke angles by simply changing the position of their fingers on the torque device. In some embodiments, the torque device allows the user to attain about 3 to about 10 stroke angles by simply changing the position of their fingers on the torque device.

In some embodiments, the torque device allows the user to attain a continuous slope, i.e. a gradual change of the radius in a tapered device by simply changing the position of their fingers on the torque device. Thus, in some embodiments, the torque device comprises an infinite number of transmission regions (i.e., a continuous gradient from a high diameter transmission region to a low diameter transmission region), and therefore an infinite number rotational radii.

In some embodiments, the torque device allows the user to attain a first stoke angle by positioning their fingers on the torque device at a first position. In some embodiments, the torque device allows the user to attain a second stoke angle by positioning their fingers on the torque device at a second position. In some embodiments, the first stroke angle is attained at a region of first transmission. In some embodiments, the second stroke angle is attained at a region of second transmission.

In some embodiments, the torque device comprising the variable speed transmission design provides a new technique for endovascular guidewire handling in order to improve navigation of tortuous anatomy and vessel selection.

The more common rolling movement in general appears to be the use of rapid back-and-forth strokes (alternating clockwise and counterclockwise strokes). However, several endovascular surgeons specifically use a technique involving consecutive strokes in the same direction (either clockwise or counterclockwise) changing the grip for every stroke, instead of the back-and-forth technique.

The reason for this preference by surgeons is unclear but it could be the result of the fact that the back-and-forth rolling movement with a stroke angle less than a complete (whole) turn, can lead to an unreachable “blind sector.” This drawback appears to be overcome by using the consecutive in the same direction, even though it is much more cumbersome and slower than the back-and-forth movement.

By allowing the user to more than one complete turn with each stoke, the embodiments of the present torque device will potentially eliminate the use of the adopted slower “same direction” technique.

In some embodiments, the torque device is contemplated for use primarily during guidewire and catheter selection in a branching tubular system. In some embodiments, the torque device can be used in the vascular system. In some embodiments, the torque device can be used in the urinary system, pulmonary system, respiratory system, gastrointestinal system and lymphatic system. In some embodiments, the torque device can be used in any system comprising a tubular structure or a tube-like structure.

In some embodiments, the goal of an endovascular procedure is advancing a guidewire to a distal target location in a tubular structure (e.g., advancing a guidewire through a blood vessel to a distal target location within the blood vessel). In some embodiments, the torque device of the present disclosure is used to help rotate the guidewire to provide direction and help advance the guidewire through narrow lesions.

In some embodiments, the torque device can be attached to a guidewire. In some embodiments, the torque device provides a “one hand loading system” that can be quickly secured to a guidewire with one hand of the user/operator. In some embodiments, the other hand of the operator/user is used to stabilize the position of the guidewire within a subject. In some embodiments, the other hand of the operator/user is used to stabilize the position of the guidewire and catheter within a subject.

As used herein, the term “subject” refers to any vertebrate including, without limitation, human, non-human primate, chimpanzee monkey, cattle, sheep, pig, goat, horse, dog, cat, mouse, rat, guinea pig, chicken, turkey, duck, geese, rabbit, cow, zebra, etc. In some embodiments, the subject is a mammal. In some embodiments, the subject is a non-mammal. In some embodiments, the subject may be a patient with a disease or a medical condition. In some embodiments, the subject may be normal without a disease or a medical condition. In some embodiments, the subject is a male or a female.

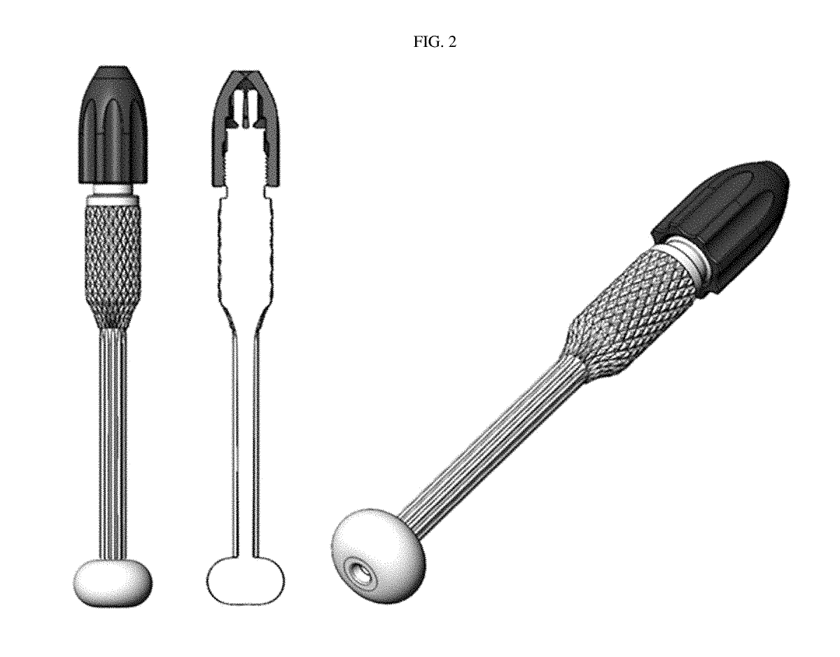

Several views of an embodiment of the novel torque device are shown in FIG. 2. A side view is shown on the left. A side bottom perspective view is shown on the right. The inner channel for the guidewire within the torque device is shown in the middle view.

In some embodiments, the torque device comprises two or more flexible prongs as shown at the top of the middle view in FIG. 2. In some embodiments, the position of the guidewire is stabilized by the flexible prongs. In some embodiments, the position of the guidewire is stabilized by the flexible prongs that sandwich/compress/grasp the guidewire in place. In some embodiments, the number of flexible prongs can range from 2 to about 10. In some embodiments, the number of flexible prongs is about 2, 3, 4, 5, 6, 7, 8, 9, or 10.

In some embodiments, the one hand loading system will allow for the ability to quickly reposition the torque device, which is often necessary throughout the endovascular procedure as the guidewire is advanced into a subject's body.

As guidewires are often of thin caliber, in some embodiments, the torque device also needs to ensure there is minimal damage to the guidewire. Thus, in some embodiments, the torque device causes minimal damage, bending and/or kinking of the guidewire thereby preventing and/or minimizing guidewire-related damage within a subject.

In some embodiments, the torque device of the present disclosure works with guidewires with a caliber range from about 0.005 inch to about 0.05 inch. In some embodiments, the torque device of the present disclosure works with guidewires with a caliber of about 0.0025, 0.005, 0.01, 0.014, 0.018, 0.025, 0.03, 0.035, 0.04, 0.045, 0.05, 0.055, 0.06, 0.065, 0.07, 0.075, 0.08, 0.085, 0.09, 0.095, or 0.1 inch, or a value within a range defined by any two of the aforementioned values. In some embodiments, the torque device of the present disclosure works with guidewires with a caliber of about 0.014 inch. In some embodiments, the torque device of the present disclosure works with guidewires with a caliber of about 0.018 inch. In some embodiments, the torque device of the present disclosure works with guidewires with a caliber of about 0.035 inch.

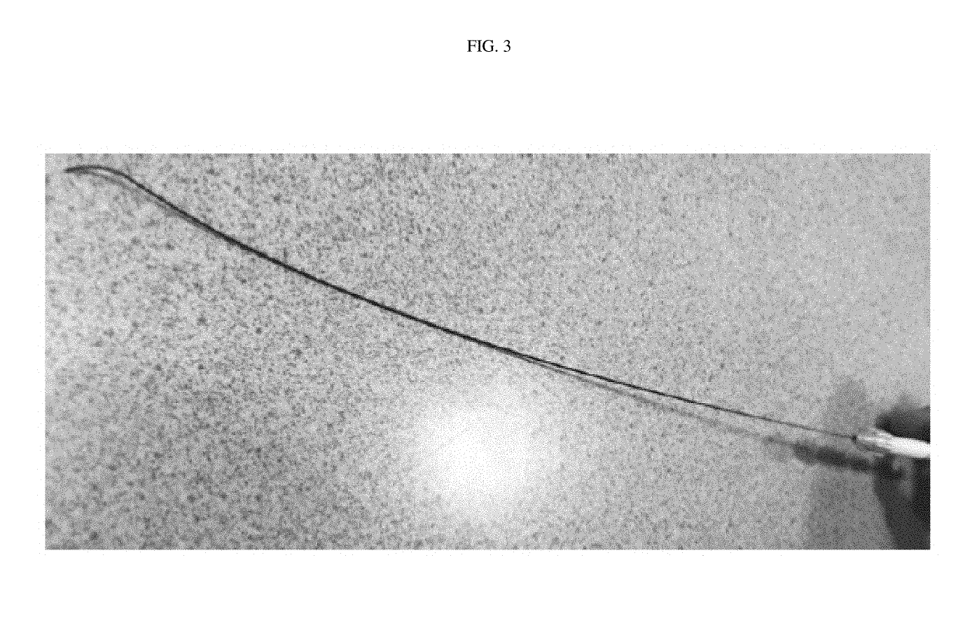

In some embodiments, the position of the guidewire is stabilized by the grasping action of the flexible prongs of the torque device to allow the guidewire to be rotated. In some embodiments, the guidewire can be rotated clockwise, counter-clockwise, or both. In some embodiments, the torque device provides a mechanism to perform rotation of the distal tip of the guidewire to pass complex lesions. The distal tip of the guidewire inserted into a subject is generally slightly curved. An example of a guidewire with a curved distal tip is shown in FIG. 3.

The challenge is to get the curved tip of a guidewire to “dance around” in the vessel to orient its way through a bifurcation in a blood vessel (e.g., a bifurcated aortic tree). In other words, the challenge is to get the curved tip of a guidewire to be oriented in the vessel such that the curved tip of the guidewire can be advanced into one or the other branch of a bifurcation in a blood vessel.

The curved tip of the guidewire (FIG. 3) helps provide direction when advancing the guidewire within a vessel. When there are multiple vessel branches or areas of narrowing, the curve on the tip helps steer the guidewire past such areas. However, it is important to be able to rotate the curved tip to be able to steer the guidewire across such areas.

In some embodiments, a torque device provides a mechanism for easier guidewire tip rotation. In some embodiments, this mechanism can comprise a spring/grasping/locking mechanism that will rotate the guidewire. In some embodiments, this mechanism can comprise a grasping mechanism that will allow the guidewire to be rotated. In some embodiments, the spring, locking, and/or grasping mechanism will make it easier for the operating surgeon to spin and/or rotate the curved tip of the guidewire during targeted vessel selection and steer the guidewire into the correct vessel (e.g., at a vessel bifurcation).

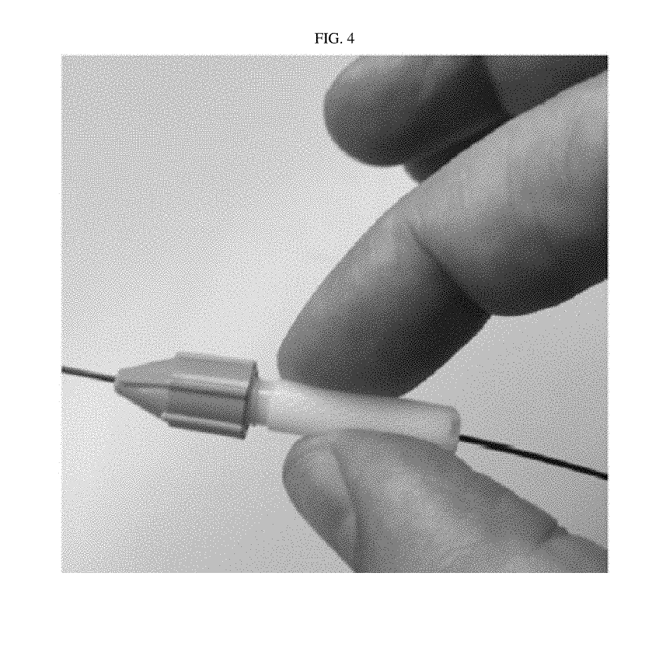

Based on the results of Example 1, the average number of turns per SL (Average 1) by each surgeon for Terumo (FIG. 4) ranged from about 0.1 to about 0.7 (Table 1). In contrast, Average 1 for an embodiment of the torque device of the present disclosure ranged from about 0.6 to about 1.6 (Table 1). In some embodiments, the average number of turns per SL for the torque device of the present disclosure ranges from about 0.3 to about 3.2. In some embodiments, the average number of turns per SL for the torque device of the present disclosure is about 0.25, 0.5, 1, 1.5, 2, 2.5, 3, 3.5, 4, 4.5, or 5, or a value within a range defined by any two of the aforementioned values.

Quotient 1, which is the ratio of Average 1 for an embodiment of the torque device of the present disclosure versus Average 1 for Terumo, ranged from about 1.7 to about 5.1 (Table 1). In some embodiments, Quotient 1 ranges from about 1.5 to about 5.5. In some embodiments, Quotient 1 is about 0.5, 1, 1.5, 2, 2.5, 3, 3.5, 4, 4.5, 5, 5.5, 6, 6.5, 7, 7.5, 8, 8.5, 9, 9.5, or 10, or a value within a range defined by any two of the aforementioned values.

The combined data for all five surgeons yielded that the average number of turns per stroke (Average 2) was about 0.36 for Terumo (Table 1). In contrast, Average 2 for an embodiment of the torque device of the present disclosure was about 1.1 (Table 1). The ratio of the Average 2 for an embodiment of the torque device of the present disclosure versus Average 2 for Terumo, was about 3 (Table 1). Thus, surprisingly, the results of this proof-of-concept experiment of Example indicates that an embodiment of the torque device of the present disclosure on average made at least three times as many turns (stroke angles) as compared to Terumo (Table 1).

In some embodiments, the average number of turns per stroke for the torque device of the present disclosure is about 3 fold higher than a current state-of-the-art torque device (e.g., Terumo). In some embodiments, the average number of turns per stroke for the torque device of the present disclosure is at least 3 fold higher than a current state-of-the-art torque device. In some embodiments, the stroke angle for the torque device of the present disclosure is greater than 3 fold higher than a current state-of-the-art torque device. In some embodiments, the stroke angle for the torque device of the present disclosure is about 3 to about 10 fold higher than a current state-of-the-art torque device. In some embodiments, the stroke angle for the torque device of the present disclosure is about 3, 4, 5, 6, 7, 8, 9, 10, 11, 12, 13, 14, or 15 fold higher than a current state-of-the-art torque device, or a value within a range defined by any two of the aforementioned values.

For more rapid search of a vessel bifurcation, it would be advantageous to be able to exceed a complete turn of the guidewire for a single stroke of the torque device. With the embodiments of the torque device of the present disclosure, a single stroke can yield greater than a complete turn of a guidewire. With the embodiments of the torque device of the present disclosure, a single stroke can yield greater than a complete turn of the guidewire with a frequency of about 40%. In contrast, a conventional state-of-the-art torque device (e.g., Terumo (FIG. 4)) greater than a complete turn of the guidewire with a frequency of less than 2%.

In some embodiments, a single stroke of the embodiments of the torque device of the present disclosure can yielded greater than a complete turn of the guidewire with a frequency of about 40% to about 75%. In some embodiments, a single stroke of the embodiments of the torque device of the present disclosure can yielded greater than a complete turn of the guidewire with a frequency of about 30, 32.5, 35, 37.5, 40, 42.5, 45, 47.5, 50, 52.5, 55, 57.5, 60, 62.5, 65, 67.5, 70, 72.5, 75, 77.5 80, 82.5, 85, 87.5, 90, 92.5, 95, 97.5, or 100%, or a value within a range defined by any two of the aforementioned values.

Clinical practitioners who perform endovascular procedures are taught and conditioned to expect a 1:1 torque relationship when rotating a guidewire using a torque device. In other words, by rotating proximal end of the guidewire by 25°, for example, with a torque device, a 25° rotation of the tip at the distal end of the guidewire is expected.

However, it has been an elusive task to develop a guidewire with favorable tracking characteristics, which maintains a 1:1 torque relationship. Guidewires, especially in tortuous anatomy, experience torsion, which dampens the response at the tip of the guidewire with each rotation of the torque device.

To overcome the dampening of guidewire tip responsiveness, hyper-torsion at the tip to get rotation is required. Hyper-torsion with current torque device designs is difficult. This is because the results of benchtop test data indicate that a one finger-stroke results in only 360 degree rotation of the guidewire tip under optimal conditions.

In some embodiments, the torque device of the present disclosure provides greater torque device control with more than 100% degree rotation compared to traditional torque devices, but at the same time maintaining the strength and precision of current devices. In some embodiments, the torque device of the present disclosure provides a guidewire with favorable tracking characteristics, which maintains a 1:1 torque relationship.

In some embodiments, the torque device of the present disclosure doubles the measured finger-stroke. Therefore, in some embodiments, the torque device of the present disclosure provides hyper-torsion to get rotation at the tip of the guidewire is provided. In some embodiments, the torque device of the present disclosure, provide hyper-torsion of guidewires by achieving at least twice the stroke angle of traditional torque devices.

In some embodiments, the torque device of the present disclosure, provide hyper-torsion of guidewires by achieving at least 3 times the stroke angle of traditional torque devices. In some embodiments, the torque device of the present disclosure, provide hyper-torsion of guidewires by achieving about 3 to about 5 times the stroke angle of traditional torque devices. In some embodiments, the torque device of the present disclosure, provide hyper-torsion of guidewires by achieving about 3, 3.5, 4, 4.5, 5, 5.5, 6, 6.5, 7, 7.5, 8, 8.5, 9, 9.5, or 10 times the stroke angle of traditional torque devices, or a value within a range defined by any two of the aforementioned values.

### Alternative Embodiments

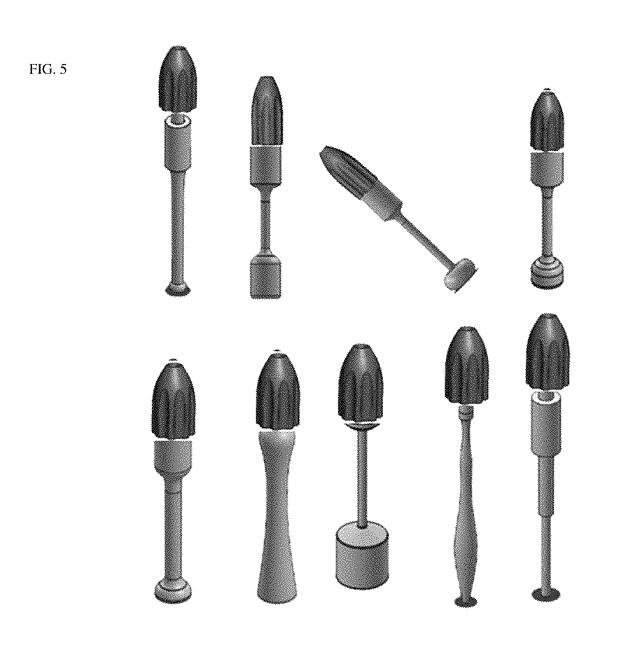

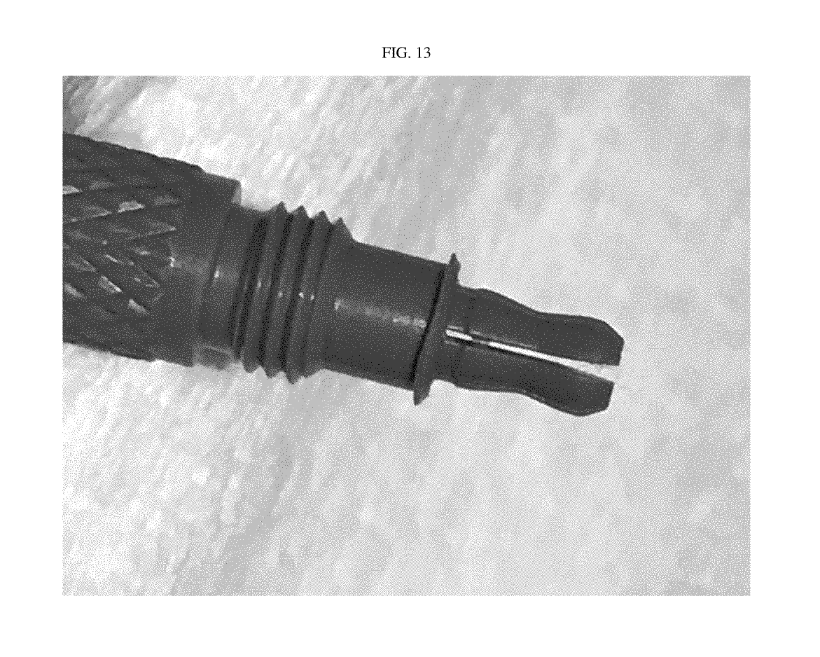

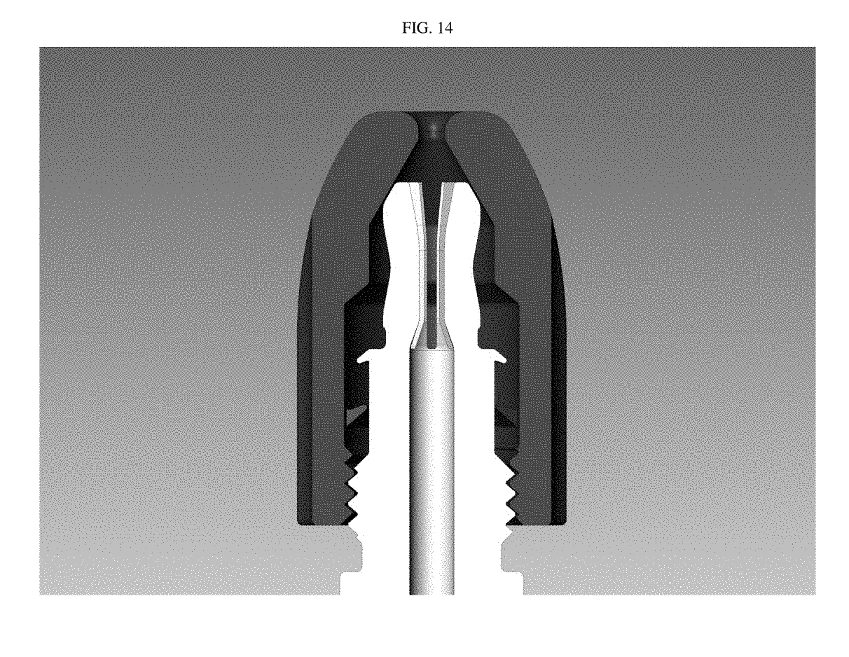

A series of alternative embodiments of the torque device of the present disclosure are contemplated. Some alternative embodiments, without limitation, are illustrated in FIG. 5, FIG. 13 and FIG. 14.

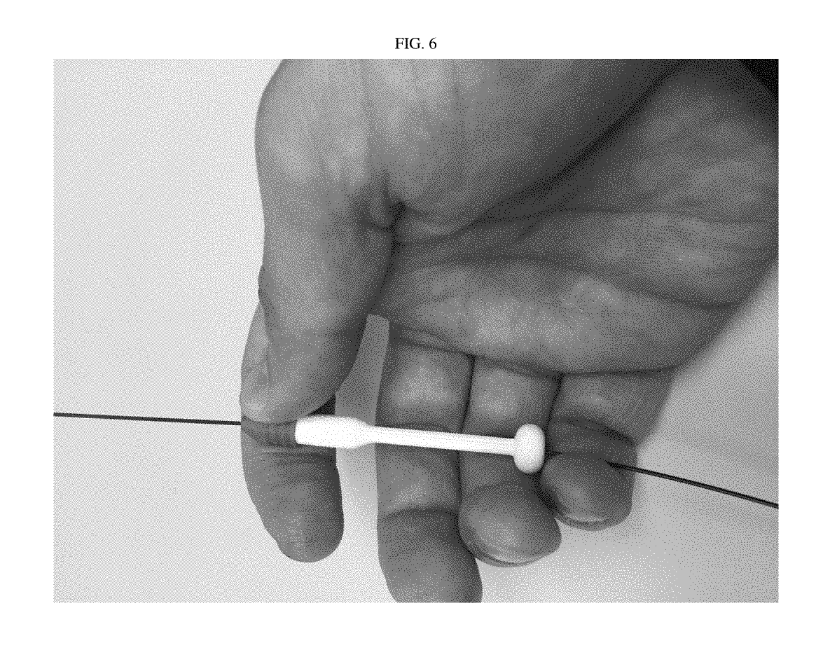

In some embodiments, the devices according to the present disclosure can be locked and unlocked with a single hand, as shown in FIG. 6. In some embodiments, an embodiment of the torque device of the present disclosure can comprise an end (distal) part that provides a grip that frees two distal phalanges to operate the locking mechanism.

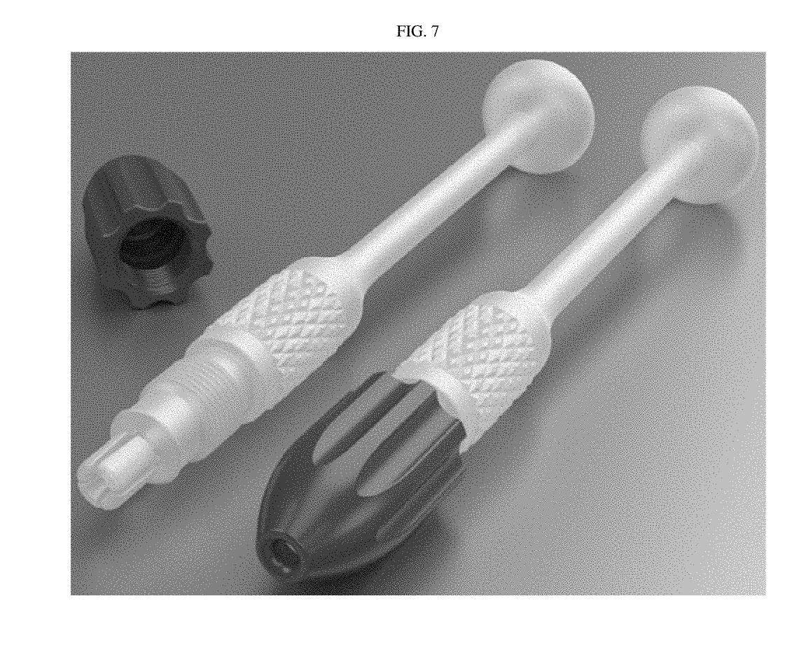

In some embodiments, the position of the guidewire is stabilized by the flexible prongs that sandwich/compress/grasp the guidewire in place when a “Cap” is locked in place. In some embodiments, an alternative of the device of the present disclosure has a “Screw Cap” design/locking mechanism (FIG. 7).

The torque device of FIG. 7 comprises two transmission regions, a first transmission region with a first diameter and second transmission region with a second diameter. The first diameter ranges from about 0.5 cm to about 1.5 cm and the second diameter ranges from about 0.1 cm to about 0.4 cm. The torque device of FIG. 7 comprises a Screw Cap design/locking mechanism. When a user loosens the Screw Cap, the guidewire is released from the grasp of and/or no longer engaged by the flexible prongs (FIG. 7; middle) allowing the torque device to slide freely along the length of the guidewire. In contrast, when the user tightens the Screw Cap, the guidewire is fixed in place by the flexible prongs grasping on and/or engaging the guidewire such that the torque device is no longer able to slide freely along the length of the guidewire. Thus, by loosening and/or tightening the Screw Cap, the user can loosen and/or tighten the Screw Cap with one hand.

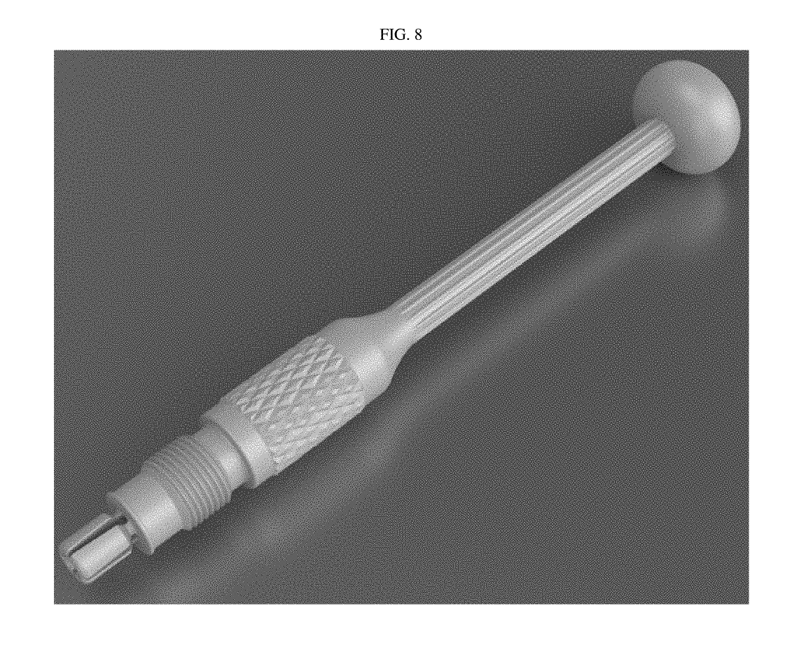

Owing to the length of the device and the presence of a “ball” at one end, this embodiment can be handled with one hand. An embodiment of the “Screw” without the locking mechanism is shown in FIG. 8. In some embodiments, the torque device allows one handed removal and/or adjustment of the guidewire and catheter within a subject.

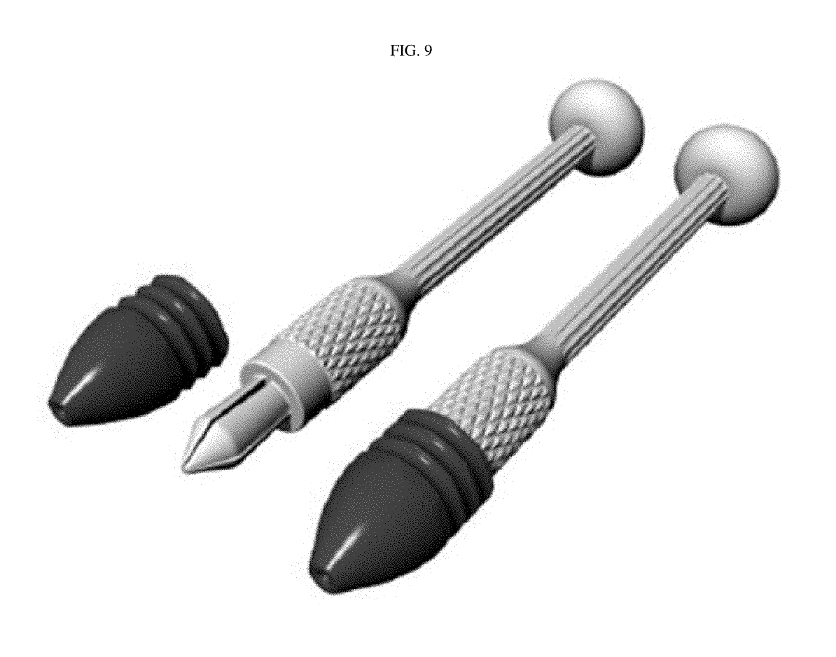

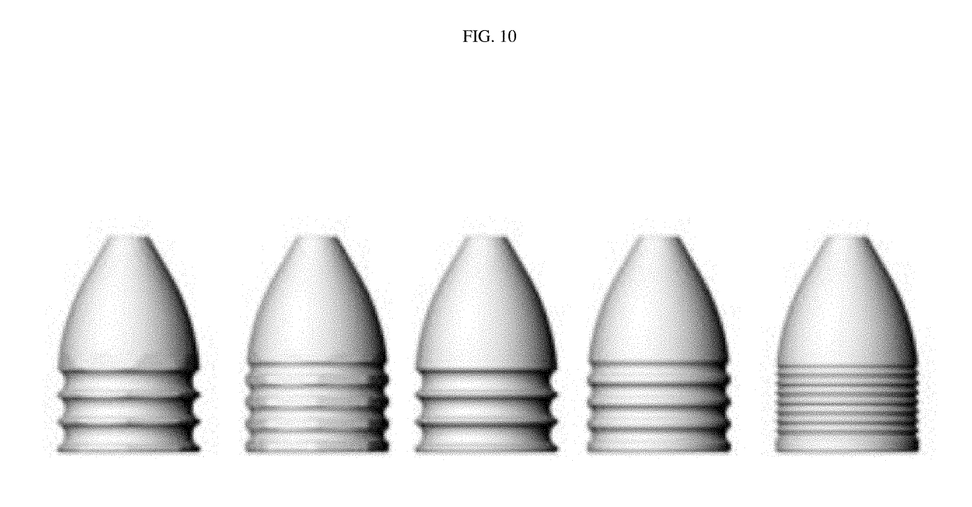

In some embodiments, an alternative of the device of the present disclosure has a “Slider Cap” design/locking mechanism (FIG. 9). In this embodiment, the “Cap” (FIG. 9, left) can slide back-and-forth over the “Slider” (FIG. 9; middle) to allow the guidewire to be released or fixed with a one hand grip. FIG. 10 shows some embodiments of the “Cap” of the “Slider Cap” design.

In some embodiments, the “Cap” is secured with a “bayonet mount.” In some embodiments, the bayonet mount is a hybrid between the Screw Cap and a Slider Cap locking mechanism.

In some embodiments, the torque device can be quickly released, with a single hand, to allow it to slide along the wire using, for example, a release button that loosens the locking mechanism.

In some embodiments, the flexible prongs of the device are short and will sandwich the guidewire such that only a small area of contact exists between the prongs and the guidewire there-between (FIG. 2; middle). In contrast, in the “flared” design (FIG. 13 and FIG. 14) the flexible prongs are longer (as compared to the length of the prongs in FIG. 2; middle), which allows for an increased area of contact between the prongs and the guidewire there-between. In some embodiment, the “flared” design will increase the contact (and thus friction) with the guidewire since the engagement of the locking mechanism will deform the entire (longer) length of the flared prongs resulting in a longer contact domain (greater contact area) between the guidewire and the prongs. It will be appreciated by one of ordinary skill in the art that the “length” of the flexible prongs can be adjusted depending on the length of the contact domain (size of the contact area) desired between the guidewire and the prongs.

It will also be appreciated by one of ordinary skill in the art that any of the flexible prong designs (and variants thereof) disclosed herein can be combined with any of the locking mechanisms (and variants thereof) disclosed herein. For example, flexible prongs with the flared design (FIG. 13 and FIG. 14) can be combined with the Screw Cap locking mechanism (FIG. 2 and FIG. 7).

In some embodiments, the “flared” design provides different “gears” to vary the rotational amplitude. In some embodiments, the “flared” design also has a “ball” at one end to allow for handling with one hand.

In some embodiments, a method for navigating a guidewire through a lumen or tubular structure (e.g., an artery) is provided. In some embodiments, the method is performed using any of the embodiments of the torque device of the present disclosure.

In some embodiments, a kit for navigating a guidewire through a lumen or tubular structure (e.g., an artery) is provided. In some embodiments, the kit comprises any of the embodiments of the torque device of the present disclosure.

### EXAMPLES

The following Examples are non-limiting and other variants contemplated by one of ordinary skill in the art are included within the scope of this disclosure.

### Example 1

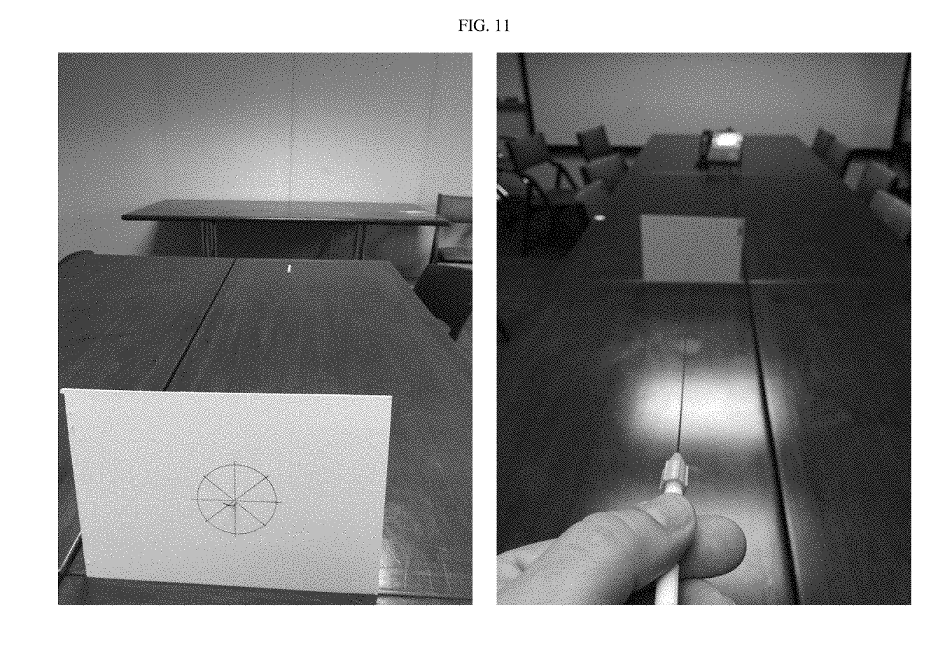

A proof-of-concept experiment was performed with five vascular surgeons. The experimental setup consisted of an embodiment of the torque device of the present disclosure, the current state-of-the-art Terumo, a guidewire with a curved tip at its distal end, and a cardboard with a circular dial on its blind side (FIG. 11). The circular dial was divided into eight sections as shown in FIG. 11 (left) in order to visualize the number of partial turns or full turns by the curved guidewire tip in response to the turning of the torque device. The embodiment of the torque device of the present disclosure was used to control a guidewire whose curved tip was on the “blindside” of the cardboard (FIG. 11, right).

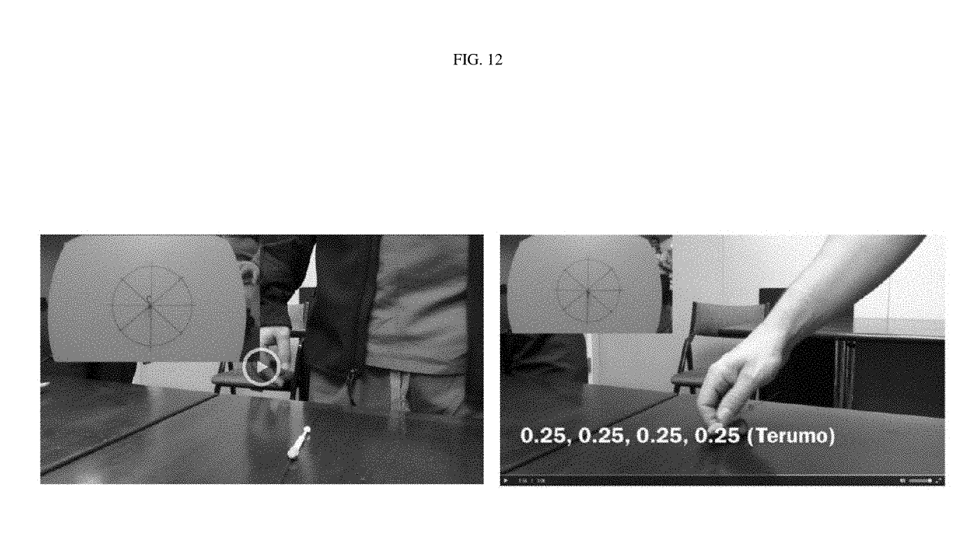

The goal was to compare the Terumo with the torque device prototype of FIG. 7 in terms of the number of turns of the guidewire for each stoke of the torque device in benchtop performance testing experiment. FIG. 12 provides two snapshots of the benchtop performance testing. Data related to the number of turns of the bent tip of the guidewire for each stroke from the benchtop performance testing experiment are provided in Table 1. The number of turns of the guidewire for each stoke of the respective torque device was counted, using a slow motion movie (thus enabling a careful determination of the angles), by visualizing the bent guidewire tip as the hand of a “clock” on the “dial” of the cardboard. FIG. 12 (right) shows an example of the turn counts for Terumo.

The results of the experiment are shown in Table 1. Phalange length on index finger (mm) is stroke length (SL) for each surgeon's most distal pointer phalange. Average 1 is the average number of turns per stroke for each of the five surgeons. Quotient 1 is the ratio of the Average 1 for the torque device of FIG. 7 versus Average 1 for Terumo. Average 2 is the average number of turns per stroke for each all five surgeons. Ratio is the ratio of the Average 2 for the torque device of FIG. 7 versus Average 2 for Terumo.

Based on the results of the experiment, the average number of turns per SL by each surgeon for Terumo (Average 1) ranged from about 0.1 to about 0.7 (Table 1). In contrast, Average 1 for the torque device of FIG. 7 ranged from about 0.6 to about 1.6 (Table 1). Quotient 1, which is the ratio of Average 1 for an embodiment of the torque device of FIG. 7 versus Average 1 for Terumo, ranged from about 1.7 to about 5.1 (Table 1).

The combined data for all five surgeons yielded that the average number of turns per stroke (Average 2) was about 0.36 for Terumo (Table 1). In contrast, Average 2 for the torque device of FIG. 7 was about 1.1 (Table 1). The ratio of the Average 2 for the torque device of FIG. 7 versus Average 2 for Terumo, was about 3 (Table 1). Thus, surprisingly, the results of this proof-of-concept experiment of indicated that the torque device of FIG. 7 on average made at least three times as many turns (stroke angles) as compared to Terumo (Table 1).

TABLE 1

Phalange
length on
index
finger

| Surgeon | (mm) | Device | No. of turns per stroke | | | | | | | | | | | | |
|---|---|---|---|---|---|---|---|---|---|---|---|---|---|---|---|
| 1 | 25.7 | Terumo | 0.6 | 0.8 | 1 | 0.3 | 0.5 | 0.5 | 0.3 | | | | | | |
| | | Torque | 2.6 | 2.8 | 2 | 1.1 | 0.9 | 1.2 | 1.2 | 1.3 | 1.4 | | | | |
| | | device | | | | | | | | | | | | | |
| | | of FIG. 7 | | | | | | | | | | | | | |
| 2 | 27.3 | Terumo | 0.1 | 0.1 | 0 | 0.1 | 0.1 | 0.1 | 0.1 | 0.1 | 0.1 | 0.13 | 0.1 | 0.1 | 0.1 |
| | | Torque | 0.9 | 0.6 | 1 | 0.5 | 0.4 | 0.4 | 0.5 | 0.5 | 0.6 | 0.6 | 0.6 | 0.8 | 0.8 |
| | | device | | | | | | | | | | | | | |
| | | of FIG. 7 | | | | | | | | | | | | | |
| 3 | 27.6 | Terumo | | | | | 0.3 | 0.3 | 0.3 | 0.3 | | | | | |
| | | Torque | 0.6 | 0.4 | 1 | 1.5 | 1.5 | 1.6 | 1.1 | 1 | | | | | |
| | | device | | | | | | | | | | | | | |
| | | of FIG. 7 | | | | | | | | | | | | | |
| 4 | 24.4 | Terumo | 0.4 | 0.3 | 0 | 0.3 | 0.3 | 0.3 | 0.1 | 0.1 | 0.2 | 0.12 | 0.1 | 0.1 | 0.1 |
| | | Torque | 1 | 0.5 | 1 | 0.5 | 0.5 | 0.9 | 1 | 1 | 0.9 | | | | |
| | | device | | | | | | | | | | | | | |
| | | of FIG. 7 | | | | | | | | | | | | | |
| 5 | 28.4 | Terumo | 0.5 | 0.5 | 1 | 0.8 | 0.8 | 0.8 | 1.1 | 0.4 | 0.8 | 0.75 | 0.8 | | |
| | | Torque | 1.3 | 1.3 | 1 | 1.1 | 1.3 | 1.5 | | | | | | | |
| | | device | | | | | | | | | | | | | |
| | | of FIG. 7 | | | | | | | | | | | | | |

| | | | | | | | | | | | Average 2 | | |
|---|---|---|---|---|---|---|---|---|---|---|---|---|---|
| | | | | | | | | | Average 1 | | (Average | Torque | |
| | | | | | | | | | (Average | | no. of | device | |
| | | | | | | | | | no. of | Quotient | turns- | of | |
| Surgeon | No. of turns per stroke | | | | | | | | turns) | 1 | Terumo) | FIG. 7 | Ratio |
| 1 | | | | | | | | | 0.54 | | 0.36 | 1.07 | 2.96 |
| | | | | | | | | | 1.65 | 3.08 | | | |
| 2 | 0.13 | 0.1 | 0.1 | 0.13 | 0.13 | 0.1 | | | 0.12 | | | | |
| | 0.75 | 0.8 | 0.6 | 0.6 | 0.9 | 0.9 | 0.8 | 0.6 | 0.64 | 5.15 | | | |
| 3 | | | | | | | | | 0.25 | | | | |
| | | | | | | | | | 1.09 | 4.35 | | | |
| 4 | 0.2 | 0.1 | 0.1 | 0.1 | 0.1 | 0.2 | 0.1 | 0.1 | 0.21 | | | | |
| | | | | | | | | | 0.76 | 3.63 | | | |
| 5 | | | | | | | | | 0.69 | | | | |
| | | | | | | | | | 1.23 | 1.77 | | | |

### Example 2

The advancement velocity for a vessel bifurcation search can be calculated as follows. Suppose that the opening radius of the bifurcated vessel is d [mm]. Then, the radius of the torque device, r, has to be less than SL/(2π). Then, it can be calculated that the advancement velocity has to be less than SL×d×f/(2πr), where f is stroke frequency (SF) [Hz]. For example, if SL=24 mm, d=4 mm, r=4 mm, f=2 Hz, the maximum velocity is 10 mm/s.

### Perspectives

In 2015, the vascular surgery department at Stanford University Hospital performed approximately 1100 endovascular procedures in the angiographic suite and operating room. In addition to vascular surgery, over 14,000 endovascular procedures were performed by cardiology, interventional radiology and interventional neurosurgery.

Thus, Stanford University Hospital alone presents an enormous pool of endovascular specialist to test the embodiments of the novel device of the present disclosure.

In addition, efficacy trials can be pursued, for example, by comparing a catheter simulator with a group of vascular residential surgeons to test the embodiments of the novel device of the present disclosure.

Furthermore, further research and development can be undertaken to improve the efficiency of endovascular procedures to make endovascular procedures, in addition to other desirable features, cost effective for subjects.

Although this disclosure is in the context of certain embodiments and examples, those skilled in the art will understand that the present disclosure extends beyond the specifically disclosed embodiments to other alternative embodiments and/or uses of the embodiments and obvious modifications and equivalents thereof. In addition, while several variations of the embodiments have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes or embodiments of the disclosure. Thus, it is intended that the scope of the present disclosure herein disclosed should not be limited by the particular disclosed embodiments described above.

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

In this application, the use of the singular includes the plural unless specifically stated otherwise. Also, the use of “comprise”, “comprises”, “comprising”, “contain”, “contains”, “containing”, “include”, “includes”, and “including” are not intended to be limiting.

As used in this specification and claims, the singular forms “a,” “an” and “the” include plural references unless the content clearly dictates otherwise.

All references cited in this disclosure are incorporated herein by reference in their entireties.

What is claimed is:

1. A torque device for navigating a guidewire through a body lumen, the torque device comprising:
an elongate body with a through channel and a locking mechanism, which together are configured to reversibly engage the guidewire within the torque device,
wherein the elongate body comprises a variable speed transmission, comprising at least first and second transmission regions, the first transmission region having a first diameter and a first longitudinal position along the elongate body, and the second transmission region having a second diameter and a second longitudinal position along the elongate body,
wherein the first transmission region is configured to induce rotation of an engaged guidewire at a first stroke angle, and the second transmission region is configured to induce rotation of an engaged guidewire at a second stroke angle, which is different from the first stroke angle.

2. The device of claims 1, wherein the at least first and second transmission regions are configured to be manually operated independently of each other.

3. The device of claim 1, wherein the stroke angle of the engaged guidewire can be varied between the first and second stoke angles by shifting between manual rotation of the first and second transmission regions, respectively.

4. The device of claim 1, wherein the at least two stoke angles allow for at least two levels of rotational control of the engaged guidewire.

5. The device of claim 4, wherein the at least two levels of rotational control comprise a first level of rotational control configured to rotate the engaged guidewire at a first rotation radius, and a second level of rotational control configured to rotate the guidewire at a second rotation radius.

6. The device of claim 5, wherein the first rotation radius is configured for a lower degree of rotation of the guidewire and the second level of rotation is configured for a higher degree of rotation of the guidewire.

7. The device of claim 6, wherein the first rotation radius is about 1 mm to about 10 mm.

8. The device of claim 6, wherein the second rotation radius is about 0.4 mm to about 5 mm.

9. The device of claim 1, wherein the device provides a stroke angle at least 2 times the stroke angle of traditional torque devices.

10. The device of claim 1, wherein the device is configured to provide a stroke angle about 3 to about 6 times the stroke angle of traditional torque devices.

11. The device of claim 1, wherein a single stroke length can yield a greater than one 360 degree rotation of the guidewire with a frequency of about 40% to about 100%.

12. The device of claim 1, wherein the guidewire has a caliber range from about 0.005 inch to about 0.05 inch.

13. The device of claim 1, wherein the locking mechanism is selected from the group consisting of a Screw Cap, Slider Cap, and bayonet mount.

14. A method of navigating a guidewire through a body lumen, the method comprising:
providing the torque device comprising:
an elongate body with a through channel and a locking mechanism, which together are configured to reversibly engage the guidewire within the torque device,
wherein the elongate body comprises a variable speed transmission, comprising at least first and second transmission regions, the first transmission region having a first diameter and a first longitudinal position along the elongate body, and the second transmission region having a second diameter and a second longitudinal position along the elongate body, and
wherein rotation of the first transmission region by manual operation is configured to induce rotation of an engaged guidewire at a first stroke angle, and rotation of the second transmission region by manual operation is configured to induce rotation of an engaged guidewire at a second stroke angle, which is different from the first stroke angle;
engaging the guidewire within the through channel by actuating the locking mechanism;
manually rotating at least one of the transmission regions thereby inducing rotation of the engaged guidewire at a stroke angle; and
navigating the guidewire through the body lumen.

15. The method of claim 14, wherein switching from the manual rotation of the first transmission region to manual rotation of the second transmission region, causes the stroke angle to shift from the first stoke angle to the second stroke angle.

16. The method of claim 14, wherein the device provides a stroke angle at least 2 times the stroke angle of traditional torque devices.

17. The method of claim 14, wherein a single stroke length can yield a greater than one 360 degree rotation of the guidewire with a frequency of at least about 40%.

18. The method of claim 14, wherein the guidewire has a caliber range from about 0.005 inch to about 0.05 inch.

19. The method of claims 14, wherein the body lumen is selected from the group consisting of a blood vessel, a duct, a tube, a tubule, and an airway.

20. A kit for navigating a guidewire through a body lumen, the kit comprising:
a guidewire, and
a torque device, the torque device comprising:
an elongate body with a through channel and a locking mechanism, which together are configured to reversibly engage the guidewire within the torque device,
wherein the elongate body comprises a variable speed transmission, comprising at least first and second transmission regions, the first transmission region having a first diameter and a first longitudinal position along the elongate body, and the second transmission region having a second diameter and a second longitudinal position along the elongate body,
wherein rotation of the first transmission region by manual operation is configured to induce rotation of an engaged guidewire at a first stroke angle, and rotation of the second transmission region by manual operation is configured to induce rotation of an engaged guidewire at a second stroke angle, which is different from the first stroke angle.
